(19) United States
(12) Patent Application Publication
(10) Pub. No.: US 2023/0263752 A1
(43) Pub. Date: Aug. 24, 2023

(54) VETERINARY COMPOSITIONS FOR THE TREATMENT AND/OR PREVENTION OF PROTOZOAN DISEASES AND METHODS OF PREPARATION THEREOF

(71) Applicant: ADIFEED SP. Z O. O., Warsaw (PL)

(72) Inventors: Henryk ROZANSKI, Krosno (PL); Hubert IWINSKI, Grodzisk Mazowiecki (PL)

(73) Assignee: ADIFEED SP. Z O. O., Warsaw (PL)

(21) Appl. No.: 18/016,181

(22) PCT Filed: Jul. 13, 2021
(86) PCT No.: PCT/IB2021/056303

§ 371 Date: Jan. 13, 2023

(30) Foreign Application Priority Data
Jul. 13, 2020 (PL) P-434641

(51) Int. Cl. A61K 31/19; A61K 47/44; A61K 47/02; A61P 33/02

(52) U.S. Cl. CPC A61K 31/19; A61K 47/44; A61K 47/02; A61P 33/02

(57) ABSTRACT

Veterinary compositions comprising an essential oil such as cedar oil, tea oil, cardamom oil, lavender oil, cubeb oil, eucalyptus oil, mint oil, juniper oil, cypress oil, lemongrass oil, spruce oil, pine oil, fir oil, Douglas fir oil, wormwood oil, caraway oil, cumin oil, patchouli oil, sage oil, Inula oil, tansy oil, angelica oil calamus oil, ginger oil, thyme oil, Origanum oil, rosemary oil, nutmeg oil, coriander oil, geranium oil, carrot seed oil, yarrow herb or flower oil are disclosed. The essential oil may be present in the form of a complex with an organic acid or a mixture of organic acids including valerian, isovalerian, lactic, butyric, acetic, propionic, formic, benzoic, pelargonic, salicylic, malonic, citric, phthalic, tartaric, oxalic, malic, shikintic, fumaric, almond, cinnamic or derivatives thereof, and a metal including molybdenum, cobalt, nickel, chromium, zinc, bismuth, copper, manganese, selenium, iron, or salts or oxides thereof. The compositions may be for the treatment and/or prevention of protozoan diseases in animals. Methods of manufacturing the veterinary compositions are also disclosed.

### CROSS-REFERENCE TO RELATED APPLICATIONS

This application is the U.S. National Phase of PCT Application No. PCT/IB2021/056303 filed on Jul. 13, 2021, which claims priority to Polish Application PL434641 filed Jul. 13, 2020, the disclosures of which are hereby incorporated by reference in their entirety.

### TECHNICAL FIELD

Veterinary compositions for the treatment and/or prevention of protozoan diseases in animals including an essential oil and methods of preparation thereof.

### BACKGROUND

The published literature indicates that some secondary plant metabolites, such as phytoncides and phytoalexins, have potential lethal and static properties against protozoa, bacteria, viruses and fungi.

Phytoncides (gr. phyton—plant; cid—syllable indicating the cidal properties) were first detected by Soviet researchers in 1928-1930. The greatest achievements in the study of phytoncides are: G. I. Nilov, B. P. Tokin (1900-1984), A. Filatov and I. Torontsev. The term and definition of phytoncides were introduced by B. P. Tokin. Phytoncides are substances secreted and excreted by higher plants (Cormophyta) with antibacterial, protozoal and fungicidal activity. Phytoncides are the equivalent of antibiotics, produced by bacteria, fungi and lichens (Tariq, S., S. Wani, W. Rasool, K Shafi, M. A. Bhat, A. Prabhakar, A. H. Shalla and M. A. Rather (2019). “A comprehensive review of the antibacterial, antifungal and antiviral potential of essential oils and their chemical constituents against drug-resistant microbial pathogens.” Microbial Pathogenesis 134: 103580).

Phytoncides are chemically very diverse. Phytoncides are in gaseous, crystalline and liquid forms. Many of them sublimate and boil at low temperatures, in the range of 30° C. According to B. M. Kozo-Polianski, the volatile fractions of phytoncides are the plant's first line of defense, while non-volatile tissue phytoncides being the second line of defense. In the literature there is a confusion regarding the terminology of chemical compounds involved in plant disease resistance processes. Related to the issue of phytoncides are the terms phytoalexins, prohibitin, inhibitin and postinhibitin. In 1960, Cruickshank and Perrin first isolated and identified the phytoalexin pisatin from Pisum sativum (pea). In 1973 J. L. Ingham published an interesting division of the factors of resistance of higher plants to infection (J. L. Ingham J. L., Disease resistance in higher plants. The concept of pre-infectional and post-infectional resistance, “Phytopath. Z.” 1973, 78, p. 314-335), wherein:

Prohibitins are metabolites that limit or completely inhibit the growth of microorganisms. They exist constantly in plant tissues in unchanging concentration, e.g. berberine (alkaloid), isoflavones, catechins.

Inhibitins are metabolites whose content in cells increases after infection, e.g. chlorogenic acid, coumarins.

Postinhibitins are substances formed from existing but phytoncidally inactive compounds, e.g. through hydrolysis, oxidation. These include cyanogenic glycosides (e.g. prunasin in Padus genus, sambunigrin in elderberries), tuliposides, glucosinolates of garlic and onions. Ingham's concept of the definition of phytoalexins is not quite right. For example, benzoic acid, coniferyl alcohol, scopoletin, resveratrol, safinol are considered as typical phytoalexins. According to Ingham's hypothesis, these substances are formed de novo, after contact with a pathogen. Meanwhile, many plants have these compounds constantly present in their chemical composition, regardless of infection, e.g. Myroxylon balsamum (L.) Harm. Prohibitins, inhibitins and postinhibitins are present in both healthy plants and plants attacked by pathogenic microorganisms, making it possible to isolate these components from plant raw materials and incorporate them into animal and human preparations. The concept of phytoalexins was developed in 1941 by K. O. Müller and H. Börger. According to this concept, a phytoalexin is a compound that inhibits the development of a pathogen (Guest, D. I. (2017). Phytoalexins, Natural Plant Protection. Encyclopedia of Applied Plant Sciences (Second Edition). B. Thomas, B. G. Murray and D. J. Murphy. Oxford, Academic Press: 124-128).

The inhibitory factor is an isolated chemical compound, a product of the host cell. Phytoalexin is a non-specific compound in its toxic effects on the pathogen. However, pathogenic organisms may exhibit varying sensitivity to these compounds. Phytoalexins include substances of diverse chemical structures, e.g. resveratrol (stilbene), cyclobrassin sulfoxide, momilactone A (diterpene), safinol (polyacetylene), scopoletin (coumarin), 7-hydroxycalamenene (sesquiterpene). Not every phytoncid is also a phytoalexin (does not satisfy Müller and Börger's rules), however every phytoalexin is a phytoncide. Attention has now turned to phytoalexins due to their strong anti-cancer properties, e.g. brassinin, resveratrol. The simplest phytoalexin is benzoic acid, which is produced by many plants when faced with pathogen intrusion into tissues. Phytoalexins, prohibitins and inhibitins have a defensive role in plants against pathogens, much like antibodies and interferon in humans and animals.

When phytoncides and later phytoalexins were discovered, studies aimed at their isolation, stabilization and application in medicine began immediately. This was largely hindered by the intensively developed research on antibiotics and sulfonamides. Cultures of antibiotic-producing bacteria and fungi and the synthesis of sulfonamides were undoubtedly simpler and cheaper, as well as more technologically accessible than phytoncides and phytoalexins. However, due to the growing problem with increasing resistance of bacteria and protozoa to commonly used chemotherapeutics, the concept of using phytoncides in medicine has now been revisited in some countries (Switzerland, Germany, USA, France). At the same time, new therapeutic properties of these compounds have been discovered, e.g. anti-atherosclerotic, hypotensive, hypoglycaemic, oncostatic and estrogenic. As early as the 1950s and 1960s, many studies showed that sulfur phytoncides have a stronger and faster antibacterial effect on Gram-positive and Gram-negative bacteria than some known antibiotics (e.g. bacitracin, neomycin). Additionally, sulphur and isosulphur phytoncides have a diastolic, cholagogic, cholepoietic and hypotensive effect (they reduce elevated blood pressure). They enhance penetration of nutrients from intestines into blood. They inhibit the growth of putrefying bacteria and pathogenic fungi. They have protozoicidal effect. They stimulate secretion of digestive juices, increase appetite, and lower cholesterol and glucose levels in blood. Ajoens (garlic oils) inhibit blood cell aggregation, preventing thrombosis. The volatile phytoncides of Asarum sp., Inula sp., marigolds, celandines, garlic or nasturtium kill mycobacteria within 3 minutes, which is faster than carbolic acid (phenol). The phytoncide stilbene resveratrol has anticancer effects, reduces the risk of myocardial infarction, improves coronary circulation and inhibits blood cell aggregation and the formation of atherosclerotic plaques. Additionally, it inhibits the growth of bacteria and fungi and lowers elevated blood glucose levels (H. Różański, J. Kilar, M. Ruda, Influence of phytoncidal plants on maintenance of deer health in organic farm animal husbandry. LXXV Scientific Meeting of the Polish Zootechnical Society. Conference materials, Poznań 2011, p. 209; E. Strzelec, R. Niżnikowski, H. Różański, M. Klockiewicz, K. Glowacz, G. Czub, A. Darkowska, K. Szymański, A. Pokrop, Effect of use of herbal feed additive on coccidian invasion level and performance traits in goats, “Annals of Warsaw University of Life Sciences—SGGW Animal Science” 2011, no. 49, p. 11-20).

With the development of analytical chemistry and phytochemistry in the 19th and 20^{th} centuries, new natural compounds with anti-parasitic activity, including protozoicidal activity, were discovered. At that time, quinine, berberine, pelletierine, allantolactone, ascaridol, santonine and others were then introduced into medical treatment, most often in the form of salts with inorganic acids (Skowroński W., Pharmacology. Published by the Polish Society of “Brotherly Help” of Students of the Academy of Veterinary Medicine, Lwów 1932, p. 183-191).

In the second half of the 20th century, sulfonamides began to be used in the treatment of protozoal diseases. Unfortunately, with the increasing use of chemical drugs, especially synthetic ones, an increase in parasite resistance to these drugs was observed. Phytoncides and phytoalexins can be a viable alternative to antibiotics and sulfonamides. These include prohibitins, inhibitins, postinhibitins and proper phytoalexins, as well as plant secondary metabolites that exert antimicrobial, disinfectant and antiseptic effects in vivo and in vitro, including compounds that are not (at the current stage of research studies) classified as phytoalexin-type resistance agents. Initially, phytoncides were defined as antibiotics produced by higher plants (B. Czerwiecki, Lexicon specificorm, FIWNIA Warsaw 1950, p. 320-323).

Strong phytoncides are produced by, among others, burnet—Sanguisorba, wormwood—Artemisia absinthium L., nettles—Urtica, beetroot—Beta, onion—Allium cepa L., corn—Zea, garlic—Allium sativum L. or Allium ursinum L., mustard—Sinapis, hogweed—Heracleum, Turkish pepper—Capsicum annuum L., Padus—Prunus padus L., hornbeam—Carpinus, poplar—Populus, oak—Quercus, jasmine—Jasminus, dogwood—Cornus, yew Taxus, radish—Raphanus, birch—Betula, horseradish—Cochlearia armoracia L., juniper—Juniperus communis L. The volatile phytoncide of garlic kills mycobacteria within 3-5 minutes, thus faster than carbolic acid. Phytoncides are a powerful factor that change the composition of microflora in the atmosphere and soil. According to B. P. Tokin and G. I. Nilov, 1 hectare of juniper tree emits 3 kg of volatile phytoncides per day; this amount is sufficient to sterilize the area of a large city (A. Danysz, Pharmacology and formulation, Ministry of Defence, Warsaw 1955, p. 41-46). The development of Soviet biologists' research on phytoncides dates back to 1928. The greatest amount of research on phytoncides was carried out by Boris Tokin, professor of biology, author of the work published in 1942 entitled “Phytoncides. “Bactericides rastitielnovo proischozdenia (phytoncides)” and the work “Phytoncides” published in 1948, as well as “Medicinal plant remedies (phytoncides)” in 1949.

In recent years, the search for new antimicrobial substances has led to a significant increase in interest in compounds of plant origin. Review papers on the use of phytoncides in recent years have been presented in: Degtyarik et al., Duka and Ardelean, Ahuja et al. (Degtyarik S. M., Slobodnitskaya, G. V., Grebneva, E. I., Benetskaya, N. A., Macksimyuk, E. V., & Bespalyi, A. V. (2017). Effect of phytoncides of plants on viability and virulence of etiologic agents of bacterial infections in fish. [symbol] [symbol] [symbol] [symbol] ; Duka, R., & Ardelean, D. (2010). Phytoncides and phytoalexins—vegetal antibiotics. Jurnal Medical Aradean (Arad Medical Journal), 13(3), 19-25; Ahuja, I., R. Kissen and A. M Bones (2012). “Phytoalexins in defense against pathogens.” Trends in Plant Science 17(2): 73-90).

A significant increase can also be observed in reviews and research papers on the use of essential oils. These cover a multidirectional application of essential oils, ranging from veterinary practice and their use in improving animal health and combating bacterial and parasitic diseases, to food preservation and the use of their therapeutic and bactericidal properties (Aleksic Sabo, V. and P. Knezevic (2019). “Antimicrobial activity of Eucalyptus camaldulensis Dehn. plant extracts and essential oils: A review.” Industrial Crops and Products 132: 413-429; Vergis, J., G. Palanisamy, R. Agarwal and A. Kumar (2013). “Essential Oils as Natural Food Antimicrobial Agents: A Review.” Critical reviews in food science and nutrition 55; Singh, A., A. K. Dwivedy, V. K. Singh, N. Upadhyay, A. K. Chaudhari, S. Das and N. K. Dubey (2019). “Essential oils based formulations as safe preservatives for stored plant masticatories against fungal and mycotoxin contamination: A review.” Biocatalysis and Agricultural Biotechnology 17: 313-317; Pateiro, M., F. J. Barba, R. Dominguez, A. S. Sant'Ana, A. Mousavi Khaneghah, M. Gavahian, B. Gómez and J. M. Lorenzo (2018). “Essential oils as natural additives to prevent oxidation reactions in meat and meat products: A review.” Food Research International 113: 156-166; Raut, J. S. and S. M. Karuppayil (2014). “A status review on the medicinal properties of essential oils.” Industrial Crops and Products 62: 250-264; Nerio, L. S., J. Olivero-Verbel and E. Stashenko (2010). “Repellent activity of essential oils: A review.” Bioresource Technology 101(1): 372-378; Deyno, S., A. G. Mtewa, A. Abebe, A. Hymete, E. Makonnen, J. Bazira and P. E. Alele (2019). “Essential oils as topical anti-infective agents: A systematic review and meta-analysis.” Complementary Therapies in Medicine 47: 102224).

Regulation (EC) No 1831/2003 retains coccidiostats and introduces histomonostats as a new category of feed additives, while establishing the withdrawal of existing antibiotics from use (and introducing on the market) as feed additives from Jan. 1, 2006, taking into account that the use of antimicrobials as growth promoters involves the risk of selecting bacterial strains resistant to drugs used in human or animal medicine. This issue was closely related to the National Program for Antibiotic Protection in Poland for 2006-2010, supervised by the Ministry of Health. According to the Program's guidelines, in order to protect the therapeutic efficacy of antibiotics, particular control should also be exercised over veterinary antibiotic therapy, the use of which should be subject to regulations analogous to those introduced in medicine. Antibiotics used in animal husbandry favour the selection and spread of resistance among microorganisms living therein.

Drug-resistant strains can move along the food chain and colonize the human gastrointestinal tract, creating reservoirs of potential pathogens, including resistance genes, e.g. Salmonella sp., Campylobacter sp., Enterococcus sp., which can then be transmitted to the etiological factors of human infections (H. Różański, W. Drymel, Herbal preparations in the prevention of malabsorption syndrome and cirrhosis in animals. Polish Poultry, part I June 2010, p. 44-46; part II July 2010, p. 28-30; part III August 2010, p. 43-44).

Some official coccidiostats have an antibiotic character with antibacterial activity, e.g. lasalocid is an ionophore polyether with anticoccidial and antibacterial activity, isolated from Streptomyces lasaliensis in 1951. Also monensin (an ionophore antibiotic) isolated in 1967 from Streptomyces cinnamonensis has coccidiostatic and antibacterial properties. Maduramicin, produced by Actinomadura rubra, additionally inhibits gram positive bacteria. These antibiotics, despite being developed for use in human medicine, have not found their way into the use therein due to their toxicity and side effects, which exceed their therapeutic value. Despite their known toxicity, the possibility of cross-resistance with other antibiotics and their accumulation in animal products if misused, EFSA has not yet gathered sufficient evidence to withdraw them from animal production. Nevertheless, discussions on this subject are ongoing and are periodically fueled by protests from various consumer and environmental organisations (Różański H., Drymel W.: Adicox as a source of phytoalexins and phytoncides. Polish Poultry. December 2010, p. 17-20).

The main problem limiting the effectiveness of antibiotics, sulfonamides and antibiotic growth promoters is antibiotic- and sulfonamide-resistance, i.e. the resistance of microorganisms to the static or lethal effect of chemotherapeutics. Acquisition of resistance by bacteria (as well as fungi and pathogenic protozoa) occurs through selection or adaptation. Resistance to microorganisms may be based on changes in their metabolism, which bypasses the pathway “blocked” by the chemotherapeutic agent, or on the production of enzymes that break down antimicrobial drugs, e.g. a penicillin-resistant strain of Staphylococcus aureus produces the enzyme penicillinase, which breaks down penicillin. This is chromosomal resistance. Resistance to chemotherapeutics (e.g. fluoroquinolones, antibiotics, sulfonamides) can be caused by inhibition of the penetration of the drug into the pathogen cell, e.g. in the case of tetracyclines. Resistance to generally used chemotherapeutics is also transmitted between microorganisms by an extrachromosomal route (plasmids). Antibiotic-sulfonamide- or fluoroquinolone resistance is a property of microorganisms, passed on to the next generation, and it is often called cross-resistance, i.e. a pathogen resistant to one chemotherapeutic agent becomes simultaneously resistant to many others, usually with a similar mechanism of action. Cross-resistance is observed e.g. to tetracyclines, partly to penicillins and cephalosporins, and to macrolide antibiotics (A. Danysz, W. Buczko, Compendium of pharmacology and pharmacotherapy, Urban and Partner, Wroclaw-Warsaw 2008).

With the introduction and uncontrolled use of an ever-increasing range of chemotherapeutic agents, and often inappropriately, there is a growing danger of fungal, viral, Actinobacteria and Chlamydia infections. The second danger of chemotherapy is enzymes that inactivate antimicrobial and antiparasitic drugs. Apart from beta-lactamase and dehydropeptidase I, enzymes inactivating aminoglycosides have been detected. Most of the antibiotics used in medicine have an adverse (immunosuppressive) effect on the immune system. Therefore, the idea of using additional immunostimulating agents in chemotherapy of infections has emerged (A. Danysz, Compendium of pharmacology and pharmacotherapy. Volumed, Wroclaw 1994, p. 110).

Many phytoncides have simultaneous antimicrobial, antiparasitic and immunostimulating effects, e.g. sesquiterpene lactones from Tanacetum, capsaicin, piperine, or latreoside from Lathraea (H. Różański, History of research and application in medicine of domestic parasitic plants of the family Scrophulariaceae and Cuscutaceae, K. Marcinkowski Medical University, Poznań 2004; W. Roeske, Outline of phytotherapy. Pharmacology and formulation of medicinal herbs, PZWL Warsaw 1955, p. 76-78; D. Korniewicz, H. Różański, Effectiveness of active substances of plant origin in pigs feeding, “Mag. Wet.”, Supl. Pigs, 2006, 22-24).

This is evidenced by recent worrying reports of the particular virulence of certain strains of E. coli and Enterococcus faecalis. E. faecalis are resistant to vancomycin (VRE), the ‘antibiotic of last resort’ produced by Amycolatopsis orientalis. Enterococcal antibiotic resistance genes find their way into other bacteria, such as staphylococci and E. coli. In the 20th century, linezolid, a synthetic antibiotic that inhibits protein synthesis in bacteria, was discovered. However, linezolid-resistant strains of vancomycin-resistant VRE have already emerged. Clinical resistance to metronidazole has been documented in protozoa, e.g. vaginal ciliates, lamblia and many anaerobic bacteria. In vitro, increasing resistance was also observed among trophozoites of dysentery creep as a result of gradually increasing doses of metronidazole (Brunton L. L., Lazo J., S., Parker K. L., Goodman and Gilman Pharmacology, volume II. Czelej Publisher, Lublin 2007, p. 1127-1129).

Phytoncides can help to address not only bacterial but also protozoal chemotherapeutic resistance.

In animal production, chemoprophylaxis has become a dangerous issue. When properly indicated, it can be useful and valuable, but in many cases it is useless or even dangerous (infection with drug-resistant bacteria and protozoa, masking of disease symptoms). Chemoprophylaxis should not be used in circumstances of zootechnical and nutritional negligence, as this undoubtedly leads to veterinary and human chemotherapy being submerged.

The greatest problem is the isolation of phytoncides from plant material and their identification and stabilization. To date, little research has been done on the antimicrobial properties of pure chemical forms of phytoncides. The antibacterial and fungistatic properties of phytoncides are identified with whole fractions of substances or extracts from medicinal plants rather than with specific compounds (R. Niżnikowski, E. Strzelec, H. Różański, M. Klockiewicz, K Glowacz, G. Czub, A. Darkowska, K Szymański, A. Pokrop: The effect of addition of phytoncides treatment to concentrate on growth performance and dairy traits in goats. IDF International Symposium on Sheep, Goat and other non-Cow Milk, Athens, May 2011; W. Drymel, H. Różański, Use of phytoalexins to improve livestock health. The Polish Branch of World's Poultry Science Association. XXII International Poultry Symposium PB WPSA, “Science for poultry practice—poultry practice for science”. Olsztyn 2010, p. 151).

AdiFeed R&D has developed a number of phytoncides-based formulations. Despite their introduction to the market, in vitro and in vivo research is still being conducted, as well as field tests on larger populations of farm animals (poultry, fur animals, pigs, ruminants). The production technology of phytoncide preparations is complicated because these compounds are labile (unstable) and reactive (they react, undergo spontaneous transformations). Some of them are lipophilic (dissolve well in organic solvents, e.g. fats, alcohols), others are hydrophilic (dissolve well in water). Therefore, many phytoncidal preparations are biphasic and take the form of emulsions.

Phytoncides belong to a diverse group of chemical compounds and hence their preparations may be alkaloid, polyphenolic, phenolic, terpene, anthraquinone, iridoid, coumarin, polyacetylene, saponin, or phenylalkylamine. Phytoncides belonging to different chemical groups can either enhance and complement each other's antimicrobial activity, or act antagonistically and cancel each other's activity.

The addition of various metals, e.g. iron, in low concentrations, enhances the antibacterial and antiparasitic activity of phytoncides. The mechanism of antiseptic and antiparasitic action may use the oligodynamic effect. It was noted that metals can inhibit the growth of microorganisms and plants if they are in the appropriate concentration for the environment. In the 19th century, the mechanism of the oligodynamic effect could not be explained. Such antimicrobial, antiseptic metals include copper, iron, silver, manganese, mercury, bismuth, tin, zinc (Różański H. Antiseptics and disinfectants used in ancient and modern medicine. Drug in Poland, vol. 14 no. 3'04, p. 66-77. Vol 13 (154) no. 10/2003, p. 68-81, vol 13 (155) no. 11/2003, p. 94-110; Penzoldt F.: Handbook of clinical pharmacology for use by physicians and students. Printed by Marya Ziemkiewiczowa, Warsaw 1891, p. 9-42).

Many of them have found lasting application in medical treatment. Soon, attention was also drawn to the “cleanliness” of metal doorknobs (e.g. brass and steel) in hospitals, which, despite being touched by numerous sick patients, do not contain active pathogenic bacteria on their surface, which, in turn, are found in large numbers on wooden objects, floors, plastics or bedding. This phenomenon is explained by the oligodynamic effect. Also, waters, including spring waters, which are rich in various metals, are very poor in bacteria. Before antibiotics and sulfonamides were used in medicine, commonly used chemotherapeutic and antiseptic preparations included bismuth, silver, mercury, iron, copper, gold, platinum, tin and zinc (Butkiewicz T.: General surgery. PZWL Warsaw 1954; p. 31-45). In the 20th century, even antibiotics (e.g. bacitracin with zinc) and sulfonamides (e.g. silver salt of sulphadiazine) were combined with metals for a more effective bacteriostatic effect (Chruściel T., Gibiński K. (ed.): Lexicon of medicaments. PZWL Warsaw 1991, p. 484-485). The dual mechanism of antimicrobial action of the silver sulfadiazine salt hinders the formation of resistant strains (Ibidem, p. 485). A similar benefit is obtained when phytoncides are combined with metals (Drymel W., Różański H.: Use of phytoalexins to improve livestock health. The Polish Branch of World's Poultry Science Association. XXII International Poultry Symposium PB WPSA, Science for poultry practice—poultry practice for science. Olsztyn 2010, p. 151; Korniewicz D., Różański H., Effectiveness of active substances of plant origin in pigs feeding. Mag. Wet., Supl.-Pigs, 2006, 22-24).

If metallic silver is added to distilled water, it acquires bactericidal properties, although the ion concentration under these conditions is only 1:20,000,000. This effect is called the oligodynamic effect, and its mechanism is not clear, despite many hypotheses (Kostowski W., Herman Z. (ed.): Pharmacology. Podstawy farmakoterapii. PZWL Warszawa 2003; 3rd edition; Volume II, p. 271; Kostowski W., Kubikowski P.: Farmakologia. Fundamentals of pharmacotherapy and clinical pharmacology. 3rd edition; PZWL Warsaw 1991, p. 740-741). One hypothesis sees the oligodynamic effect in disrupting the distribution of ionic charges within cell membranes, disrupting the polarity of the cell. Many metals also destabilize (by attaching to) the structure of key proteins (enzymes, channel proteins) and nucleic acids.

Protozoan diseases of animals and humans cause significant morbidity and mortality worldwide. The use of chemotherapeutics to treat protozoan infections has proven to be problematic due to increasing drug resistance, variable efficacy between strains or species and toxicity. There is a strong need to find new effective solutions to treat these diseases.

During the analysis of literature reports and the state of the art, it was observed that published studies are mainly based on the analysis of protozoan inhibitory properties (IC₅₀ and IC₁₀₀) over a period of 24 to 72 hours. A much smaller percentage of researchers performed analyses with respect to the lethal dose for protozoa.

The use of plant materials, in the treatment of parasitoses, is common in natural and traditional medicine. Previous studies have shown that medicinal plants contain active compounds that exhibit potent activity against protozoa. Examples of commonly used natural antiparasitic agents of plant origin are quinine—an alkaloid from the bark of the quinine tree, and artemisinin—a sesquiterpene from Artemisia annua and Artemisia indica (Hygeia Public Health 2014, 49(3): 442-448).

A number of published scientific experimental reports demonstrate the inhibition of protozoan growth (in vitro and in vivo) by the plant extracts and secondary metabolites selected from: essential oils, alkaloids, phenolic compounds (Natural products as sources of antiprotozoal drugs. Current Opinion in Anti-infective Investigational Drugs 2000; 2, 47-62).

Copper-based complexes are known to act through interactions with protozoan DNA, e.g. Trypanosoma cruzi. Becco et al. demonstrated an inhibitory effect on 50% of the population (IC₅₀) for the compounds they synthesised at 3.9±1.5 to 11.3±3.8 μM, compared to the drug Nifurtimox (6 μM). The IC₁₀₀ value was achieved for the analysed compounds at concentrations >20 μM (Becco, L., Rodriguez, A., Bravo, M. E., Prieto, M. J., Ruiz-Azuara, L., Garat, B., Moreno V., Gambino, D. (2012). New achievements on biological aspects of copper complexes Casiopeinas®: Interaction with DNA and proteins and anti-Trypanosoma cruzi activity. Journal of inorganic biochemistry, 109, 49-56).

Other researchers have proposed vanadium complexes with 2,2′-bipyridine or dipyridine [3,2-a:2′,3′-c]phenazine, and salicylaldehyde semicarbazide or its derivative, 5-bromosalicylaldehyde semicarbazide. Similar to previous researchers, nifurtimox was used as the reference substance. They obtained IC₅₀ results for four variants of the complexes in the range of 13-84 μM (Benitez, J., L. Guggeri, I. Tomaz, G. Arrambide, M. Navarro, J. Costa Pessoa, B. Garat and D. Gambino (2009). “Design of vanadium mixed-ligand complexes as potential anti-protozoa agents.” Journal of Inorganic Biochemistry 103(4): 609-616).

Similar analyses were conducted by two teams of researchers: Martins et al. and Paixão et al. In their study, they focused on using copper ions to create complexes showing properties against Trypanosoma cruzi. The first group of researchers successfully used commonly used antibiotics (levofloxacin and sparfloxacin) to create the complexes (Martins, D. A., Gouvea, L. R., Batista, D. D. G. J., Da Silva, P. B., Louro, S. R., Maria de Nazaré, C. S., & Teixeira, L. R. (2012). Copper (II)—fluoroquinolone complexes with anti-Trypanosoma cruzi activity and DNA binding ability. BioMetals, 25(5), 951-960). Paixão et al., like Benítez et al., created complexes of the general formula [Cu(N_O) (N_N)]²⁺, using 2-methoxybenzhydrazide, 4-methoxybenzhydrazide and three α-diimine ligands: 1,10-phenanthroline, 2,2′-bipyridine and 4-4′-dimethoxy-2-2′-bipyridine (Paixão, D. A., Lopes, C. D., Carneiro, Z. A., Sousa, L. M., de Oliveira, L. P., Lopes, N. P., Pivatto M., Chaves J. D. S., de Almeida M. V., Ellena J., Moreira M. B., Netto A. V. G., de Oliveira R. J., Guilardi S., de Albuquerque S., Guerra W Moreira, M. B. (2019). In vitro anti-Trypanosoma cruzi activity of ternary copper (II) complexes and in vivo evaluation of the most promising complex. Biomedicine & Pharmacotherapy, 109, 157-166).

Sulfoaminoamide complexes with copper and zinc 8-aminoquinoline groups showed efficacy against pathogenic strains of Leishmania braziliensis, chagasi and Trypanosoma cruzi. Their lowest IC₅₀ was determined to be 0.35 mM (about 0.034%) under laboratory conditions (Everson da Silva, L., Teixeira, D. S. J., Nunes Maciel, E., Korting Nunes, R., Eger, I., Steindel, M., & Andrade Rebelo, R. (2010). In vitro antiprotozoal evaluation of zinc and copper complexes based on sulfonamides containing 8-aminoquinoline ligands. Letters in Drug Design & Discovery, 7(9), 679-685).

Other synthetic metal complexes i.e. manganese, cobalt, nickel in the form of 4′-(2-ferrocenyl)-2,2′:6″2″-terpyridinium derivatives under in-vitro conditions were very effective at a concentration of 1.1 mM against Plasmodium falciparum. The authors demonstrated the effectiveness of mixtures of manganese, iron, cobalt, nickel and copper salts (Al-Khodir, F. A. I., & Refat, M. S. (2017). Investigation of coordination ability of Mn (II), Fe (III), Co (II), Ni (II), and Cu (II) with metronidazole, the antiprotozoal drug, in alkaline media: Synthesis and spectroscopic studies. Russian Journal of General Chemistry, 87(4), 873-879).

The possibility of effective complex formation by a commonly used antibiotic with strong antiprotozoal activity (Metronidazole), with metals including Mn(II), Fe(III), Co(II), Ni(II), and Cu(II) has also been demonstrated (Al-Khodir, F. A. I. and M. S. Refat (2017). “Investigation of coordination ability of Mn(II), Fe(III), Co(II), Ni(II), and Cu(II) with metronidazole, the antiprotozoal drug, in alkaline media: Synthesis and spectroscopic studies.” Russian Journal of General Chemistry 87(4): 873-879).

Enhancement of the activity of antiprotozoan metal ions of copper and zinc in synthetic organic complexes of imidazopyridines and diarylpiperidines has also found its patent protection. Thus, the patents U.S. Pat. No. 6,291,480 B1 and US20060178358 B1 relates to the activity of diarylpyridyl derivatives against Toxoplasma gondii, Trypanosoma cruzi and Emeria species: tenella, acervulina, necatrix, brunetti maxima. US20110207701 A1 is another example of antiprotozoal applications of metal complexes including copper and low molecular weight bioorganic compounds.

Previous studies on antimicrobial properties have shown very strong effects of essential oils. Escobar, P et al. conducted studies on the antimicrobial properties of five plants of the genus Lippi. They analysed the extracted oils in terms of inhibition of the development of protozoa on Trypanosoma cruzi and Leishmania chagasi, with reference to nifurtimox. They obtained IC₅₀ values from 4.4 to >100 μg/ml, while for nifurtimox the value was 0.3-0.4 μg/ml (Escobar, P., Milena Leal, S., Herrera, L. V., Martinez, J. R., & Stashenko, E. (2010). Chemical composition and antiprotozoal activities of Colombian Lippia spp essential oils and their major components. Memórias do Instituto Oswaldo Cruz, 105(2), 184-190).

Similarly, the application WO2008101131 A1 relates to a composition for killing or repelling ectoparasites and/or pests, comprising at least 3% Lippia javanica essential oil and at least one other essential oil.

Another group of researchers demonstrated the effect of essential oils from Annona coriacea on Trypanosoma cruzi and different Leishmania species (Leishmania (L.): amazonensis, braziliensis, chagasi, major). Two compounds commonly used for leishmaniasis were used for comparative analysis: pentamidine and benznidazole. The values obtained for the essential oils (39.93-261.20 μg/mL) were significantly higher than those for the reference drugs tested (0.06-022 μg/mL and 45.02 μg/mL, respectively) (Siqueira, C. A. T., J. Oliani, A. Sartoratto, C. L. Queiroga, P. R. H. Moreno, J. Q. Reimão, A. G. Tempone and D. C. H. Fischer (2011). “Chemical constituents of the volatile oil from leaves of Annona coriacea and in vitro antiprotozoal activity.” Revista Brasileira de Farmacognosia 21: 0-0).

Perez et al. in their review paper collected information on the antiprotozoal properties, IC₅₀ (Giardia lamblia, Trichomonas vaginalis, Leishmania sp, Trypanosoma cruzi) for thyme, garlic, basil, lavender, tea or yarrow oils, among others. They demonstrated antiprotozoal properties of essential oils in a very wide range of concentrations, from 8.3 ng/ml to 8 mg/ml (Pérez, S., M. Ramos-Lopez, E. Sánchez-Miranda, M. Fresán-Orozco and J. Pérez-Ramos (2012). “Antiprotozoa activity of some essential oils.” Journal of medicinal plant research 6: 2901-2908).

Monzote et al. collected literature reports on the antiparasitic properties of essential oils between 1988 and 2012. They present a significant increase in the interest and amount of research on the use of essential oils against protozoa (Monzote, L., O. Alarcón and W. Setzer (2012). “Antiprotozoal Activity of Essential Oils.” Agriculturae Conspectus Scientificus 77: 167-175).

Moon et al, in their research paper, presented the protozoicidal properties of two lavender oils against Giardia duodenalis, Trichomonas vaginalis and Hexamita inflata. They demonstrated that a concentration of 0.1% of lavender oil has a protozoicidal effect against the analysed protozoa (Moon, T., J. Wilkinson and H. Cavanagh (2006). “Antiparasitic activity of two Lavandula essential oils against Giardia duodenalis, Trichomonas vaginalis and Hexamita inflata.” Parasitology research 99: 722-728).

The application EP2070427 A1 relates to the use of at least one essential oil compound selected from the group consisting of cinnamaldehyde, 2-decenal and nerolidol as, or in the preparation of, a histomonastat. Preferably, at least one essential oil compound is additionally combined with at least one compound selected from the group consisting of p-cymene, thymol, salicylaldehyde, tea tree oil, peppermint oil, cuminaldehyde, cinnamic acid, cinnamic alcohol, farnesal and farnesylacetone.

The application EP2119363 A2 relates to an antimicrobial composition, based on plant essential oils, of enhanced anti-microbial effectiveness, comprising: at least two plant essential oils as a major component; and a small but antimicrobial enhancing effective amount of an enhancer selected from the group consisting of polyionic organic enhancers (e.g. polyetheyleneimine) and polyionic inorganic enhancers (e.g. sodium tripolyphosphate, sodium hexametaphosphate).

The application US20140106012 AA relates to a composition comprising: an essential oil selected from the group consisting of anise oil, rosemary oil, calendula oil, tea tree oil, sassafras oil, quassia oil, cinnamon oil, clove oil, eucalyptus oil, lavender oil, peppermint oil, or combinations thereof; from about 10% to about 30% (v/v) isopropyl alcohol; from about 30% to about 50% (v/v) isopropyl myristate; between about 5% and about 20% (v/v) of a silicone oil; and from about 5% to about 25% (v/v) capric/caprylic trigylceride.

The document EP1512409 B1 relates to an aqueous composition for the treatment of headlice and their eggs, which includes as its active ingredients at least one essential oil. The composition further includes an infusion of: dried peppermint leaves, a tea, and garlic. A method of manufacture of said composition for the treatment of headlice is disclosed and includes the steps of: making an infusion of peppermint leaves, tea and garlic in boiling water and allowing to cool, adding essential oils to the cooled infusion and then mixing the cooled infusion with surfactants and thickening agents to form a gel.

Patent EP1089745 B1 relates to the use of extract of oregano or a metabolic product of the extract of oregano for the manufacture of a medicament for reducing or eliminating intestinal amoeba selected from the group consisting of Entamoeba hartmanni, Blastocystis hominis, Endolimax nana, and Entamoeba histolytica in humans in need thereof wherein such a medicament is adapted for administration in the form of an emulsified, sustained release tablet comprising carvacrol as an active ingredient.

US20140037698 AA (EP2666364 B1) relates to an additive for animal feed comprising the combination of a salt of an organic acid with at least one active ingredient of plant origin. This combination is partially coated with vegetable oils and/or fats. The active ingredients of plant origin comprise essential oils selected from the group consisting of ginger, pipeline, oregano, thymol, carvacrol, cinnamaldehyde, garlic, and combinations thereof. The organic acid is selected from the group consisting of butyric, propionic, formic, lactic, citric, lauric, capric, caprylic, caproic, and acetic. The disclosed food additive has antiprotozoal properties.

### SUMMARY

A new composition for controlling protozoa with -cidal properties.

A veterinary composition comprising an essential oil is disclosed. The essential oil comprises cedar oil, tea oil, cardamom oil, lavender oil, cubeb oil, eucalyptus oil, mint oil, juniper oil, cypress oil, lemongrass oil, spruce oil, pine oil, fir oil, Douglas fir oil, wormwood oil, caraway oil, cumin oil, patchouli oil, sage oil, Inula oil, tansy oil, angelica oil calamus oil, ginger oil, thyme oil, Origanum oil, rosemary oil, nutmeg oil, coriander oil, geranium oil, carrot seed oil, yarrow herb or flower oil, wherein the said essential oil is present in the form of a complex with an organic acid or a mixture of organic acids. The acids may comprise valerian, isovalerian, lactic, butyric, acetic, propionic, formic, benzoic, pelargonic, salicylic, malonic, citric, phthalic, tartaric, oxalic, malic, shikimic, fumaric, almond, cinnamic or derivatives thereof, and a metal selected from the group comprising molybdenum, cobalt, nickel, chromium, zinc, bismuth, copper, manganese, selenium, iron, their salts or oxides. The composition may be for use in the treatment and/or prevention of protozoan diseases in animals.

Preferably, the mixture of organic acids is a mixture of acetic acid, propionic acid, lactic acid and formic acid.

Preferably, the acids in the mixture of organic acids are mixed at a ratio of 1:1:1:1.

A method of manufacturing a veterinary composition containing an essential oil for treating and/or preventing protozoan diseases in animals is also disclosed. The method comprises the following steps:
a) mixing at least one essential oil comprising cedar oil, tea oil, cardamom oil, lavender oil, cubeb oil, eucalyptus oil, mint oil, juniper oil, cypress oil, lemongrass oil, spruce oil, pine oil, fir oil, Douglas fir oil, wormwood oil, caraway oil, cumin oil, patchouli oil, sage oil, Inula oil, tansy oil, angelica oil calamus oil, ginger oil, thyme oil, Origanum oil, rosemary oil, nutmeg oil, coriander oil, geranium oil, carrot seed oil, yarrow herb or flower oil, with an organic acid or a mixture of organic acid comprising valerian, isovalerian, lactic, butyric, acetic, propionic, formic, benzoic, pelargonic, salicylic, malonic, citric, phthalic, tartaric, oxalic, malic, shikimic, fumaric, almond, cinnamic or derivatives thereof in a proportion by weight of 80:1 to 1:80;
b) adding a catalyst to the mixture from step a);
c) adding a metal comprising molybdenum, cobalt, nickel, chromium, zinc, bismuth, copper, manganese, selenium, iron, their salts or oxides;
d) heating the mixture obtained with the catalyst and the metal to the boiling point and continuing the heating at boiling point under reflux for 20 to 120 minutes;
e) allowing the reaction product to cool for 10 to 24 hours;
f) filtering the cooled reaction product.

Preferably, in step a) the essential oil is mixed with an organic acid or mixture of organic acids in a ratio of 1:1 by weight.

Preferably, the organic acid mixture used in step a) is a mixture of acetic acid, propionic acid, lactic acid and formic acid.

Preferably, the acids in the mixture of organic acids are mixed in a ratio of 1:1:1:1.

Preferably, in step b) a mixture of cobalt sulphate, ammonium molybdate and manganese chloride or sulphate is used as a catalyst.

The compositions may provide the following favourable effects:
the protozoicidal properties of the composition ensure complete elimination of infection;
the composition is effective in low concentrations;
the composition exhibits a broad spectrum of action—i.e. it exhibits good killing activity against many species of protozoa;
the composition can be an alternative to protozoal chemotherapeutics (Antiprotozoal), such as, for example, metronidazole, albendazole, tinidazole, amprolium, lasalocid, salinomycin, robenidine, nicarbazin, monensin, decoquinate, diclazuril, or it can also be an additive to antiprotozoal chemotherapeutics, reducing the risk of resistance to a given drug.

### DETAILED DESCRIPTION

The compositions and methods are set out in detail in the following examples, wherein all tests and experimental procedures described below were carried out using commercially available test kits, reagents and devices, following the recommendations of the manufacturers of the kits, reagents and devices used, unless otherwise expressly indicated. All test parameters were measured using standard, well-known methods used in the field.

All raw materials used in the study are approved for both animal and human nutrition by the relevant directives and authorities. The selection of raw materials was made on the basis of Codex Alimentarius, i.e. the Codex Alimentarius established by FAO and WHO, Der Deutsche Arzneimittel-Codex (DAC), guidelines of the European Food Safety Authority (EFSA) and Regulation (EC) No 1831/2003 of the European Parliament and of the Council of 22 Aug. 2003 on additives for use in animal nutrition. In addition, the essential oils used in the study met the requirements of the European Pharmacopoeia, the Swiss Pharmacopoeia and Der Deutsche Arzneimittel-Codex (DAC).

For in vitro tests of antiprotozoal activity of the composition five reference organisms representing taxonomic groups to which pathogenic protozoa belong were selected, i.e:
Amoeba proteus—Chaos diffluens—a protozoan of the order Euamoebida, belonging to fifth supergroup of the Amoebozoa, living in waters.
Paramecium caudatum—a slipper animalcule representing the Ciliata orachs, living in waters.
Gregarina blattarum—gregarine isolated from cockroaches, representing the phylum Apicomplexa, living in the digestive tracts or body cavities of invertebrates.
Euglena gracilis—a protozoan living in water, representing the flagellates—Mastigophora, family Euglenaceae.
Trichomonas hominis—a protozoan living in the human colon, representing the Trichomonadidae.

Amoeba, Paramecium, Trichomonas and Euglena were observed under a microscope on watch glasses with viscose wool fibers (to facilitate observation) in a drop of water from the culture they came from. Different concentrations of the test compositions were introduced to the test samples, establishing an LD₅₀ dose (50% mortality) and an LD₁₀₀ dose (100% mortality). In all cases, 4-fold replicates of the test were used together with a blank test.

The gregarines were isolated from the cockroaches and, after being placed on a watch slide, were treated with the products at different concentrations in Ringer's solution. Each sample contained ten individuals. The lethal concentration of the substance for 50% and 100% of the individuals (LD₅₀, LD₁₀₀) within 3 minutes was determined. Isolation of gregarines from cockroaches was performed on the basis of the method of isolation of gregarines from beetles proposed by J. Moraczewski (Moraczewski J.: Exercises in the zoology of invertebrates. 1st Edition, PWN, Warsaw 1974, p. 29-31, p. 285-292).

Identification of individual protozoa was made on the basis of their descriptions and drawings after W. A. Dogiel and J. Hempel-Zawitkowska (Dogiel W. A.: Invertebrate zoology. 3rd edition, National Agricultural and Forest Publishing House 1972; Hempel-Zawitkowska J., Galka B., Kalińska B., Kamionek M., Komosińska H., Pezowicz E. Podsiadło E., Sulgostowska T.: Zoology for agricultural universities. Scientific Publishing House PWN 2008).

The compositions, negative controls, and positive controls were dissolved in an aqueous solution of polysorbate 80 (0.05%) before application to a watch slide. No lethal effect of polysorbate 80 at the above concentration was observed.

### EXAMPLE 1

### A Combination of an Eucalyptus Oil (Eucalyptus globulus Labill.) with an Acid a Ratio of 1:1 and with Copper or Zinc

In this non-limiting example, the following two compositions were prepared:
a) composition I—i.e., a composition of eucalyptus oil with an acetic acid and copper
b) composition II—i.e., a composition of eucalyptus oil with an acetic acid and zinc

In order to prepare composition I, 100 ml of acetic acid, 0.6 g of catalyst and 5 g of copper carbonate were added to 100 ml of eucalyptus oil. A mixture of cobalt sulfate, ammonium molybdate and manganese chloride mixed in a ratio of 1:1:1 was used as a catalyst in this example. The mixture was heated at the boiling point, until the color changed, under reflux for 120 minutes. The mixture was then left to cool at a room temperature for 24 hours to obtain a clear solution. The solution may be a one, two or three-phase solution. After this time, the reaction product was filtered through filter paper. Composition II was prepared analogously to composition I, except that 5 g of zinc carbonate was used as the metal. Compositions I and II were then analysed for their antiprotozoal properties. For this purpose, both compositions were diluted: 0.001% to 1%, after which the following protozoa were placed in each dilution:
Amoeba proteus—Chaos diffluens—a protozoan of the order Euamoebida, belonging to fifth supergroup of the Amoebozoa, living in waters.
Paramecium caudatum—a slipper animalcule representing the Ciliata orachs, living in waters.
Gregarina blattarum—gregarine isolated from cockroaches, representing the phylum Apicomplexa, living in the digestive tracts or body cavities of invertebrates.
Euglena gracilis—a protozoan living in water, representing the flagellates—Mastigophora, family Euglenaceae.
Trichomonas hominis—a protozoan living in the human colon, representing the Trichomonadidae.

Individual acid, catalyst solution, copper carbonate solution, zinc carbonate solution and eucalyptus oil (Eucalyptus globulus Labill.) were additionally analysed for protozoal properties. The control antiprotozoal substances used were CH—chloramphenicol and M—metronidazole. The tested preparations were dissolved in an aqueous solution of polysorbate 80 (0.05%) before being applied to a watch glass. No killing effect of polysorbate 80 in the above mentioned concentration was observed. Observation under a fluorescence microscope with phase contrast was carried out. Protozoicidal activity was considered effective when the death of 50% and 100% of individuals occurred within 3 minutes. The results obtained from the protozoal activity test are presented in Table 1. The results of the analysis showed that the killing and static activity in the compositions and after the reaction was higher than that of the individual substances in the reaction mixtures and complexes. Compositions I and II show many times stronger (potentiation) protozoal activity than each of these components separately. All the ingredients used in the compositions according to the invention are approved for both animal and human nutrition by the relevant directives and authorities, which, combined with their high efficacy, allows their use in the treatment and/or prevention of parasitoses in animals, caused by protozoa, in particular histomonadiasis (caused by Histomonas meleagridis), coccidiosis (caused by Eimeria), cryptosporidiosis (caused by Cryptosporidium), trichomonadiasis (caused by Trichomonas), babesiosis (caused by Babesia), or amoebiasis (caused by Amoeba).

TABLE 1
LD₅₀, LD₁₀₀ values for compositions I and II, determined for selected protozoa.

| Sample | | | | Catalyst | Copper carbonate | Zinc carbonate | Eucalyptus oil | Composition | Composition |
|---|---|---|---|---|---|---|---|---|---|
| Protozoa | CH* | M** | Acid | solution | solution | solution | (Eucalyptus globulus Labill.) | I | II |
| Euglena | LD₅₀: | LD₅₀: | LD₅₀: | LD₅₀: | LD₅₀: | LD₅₀: | LD₅₀: | LD₅₀: | LD₅₀: |
| gracilis | 0.05% | LD₁₀₀: | 0.5% | 0.05% | 0.15% | 0.1% | 0.1% | 0.004% | 0.002% |
| | LD₁₀₀: | | LD₁₀₀: | LD₁₀₀: | LD₁₀₀: | LD₁₀₀: | LD₁₀₀: | LD₁₀₀: | LD₁₀₀: |
| | 0.1% | | 1.1% | 0.1% | 0.25% | 0.3% | 0.2% | 0.008% | 0.004% |
| Gregarina | LD₅₀: | LD₅₀: | LD₅₀: | LD₅₀: | LD₅₀: | LD₅₀: | LD₅₀: | LD₅₀: | LD₅₀: |
| blattarum | LD₁₀₀: | 0.13% | 0.9% | 0.07% | 0.12% | 0.2% | 0.08% | 0.004% | 0.003% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.3% | 1.1% | 0.3% | 0.37% | 0.4% | 0.1% | 0.005% | 0.006% |
| Amoeba | LD₅₀: | LD₅₀: | LD₅₀: | LD₅₀: | LD₅₀: | LD₅₀: | LD₅₀: | LD₅₀: | LD₅₀: |
| proteus | 0.07% | 0.3% | 0.6% | 0.05% | 0.09% | 0.15% | 0.05% | 0.003% | 0.005% |
| | LD₁₀₀: | LD₁₀₀: | LD₁₀₀: | LD₁₀₀: | LD₁₀₀: | LD₁₀₀: | LD₁₀₀: | LD₁₀₀: | LD₁₀₀: |
| | 0.15% | 0.5% | 1% | 1% | 0.17% | 0.25% | 0.07% | 0.006% | 0.006% |
| Paramecium | LD₅₀: | LD₅₀: | LD₅₀: | LD₅₀: | LD₅₀: | LD₅₀: | LD₅₀: | LD₅₀: | LD₅₀: |
| caudatum | 0.001% | LD₁₀₀: | 0.8% | 0.8% | 0.35% | 0.3 | 0.1% | 0.002% | 0.006% |
| | LD₁₀₀: | | LD₁₀₀: | LD₁₀₀: | LD₁₀₀: | LD₁₀₀: | LD₁₀₀: | LD₁₀₀: | LD₁₀₀: |
| | 0.006% | | 1.25% | 1.25% | 0.5% | 0.5% | 0.3% | 0.006% | 0.008% |
| Trichomonas | LD₅₀: | LD₅₀: | LD₅₀: | LD₅₀: | LD₅₀: | LD₅₀: | LD₅₀: | LD₅₀: | LD₅₀: |
| hominis | LD₁₀₀: | 0.05% | 0.8% | 0.9% | 0.1% | 0.25% | 0.09% | 0.045% | 0.025% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.1% | 1% | 1.1% | 0.3% | 0.4% | 0.1% | 0.075% | 0.035% |

*CH—chloramphenicol;
**M—metronidazole

### EXAMPLE 2

### A Combination of an Eucalyptus Oil (Eucalyptus globulus Labill.) with an Acid a Ratio of 1:1 and with Manganese

In order to prepare composition III, 100 ml of propionic acid, 0.6 g of catalyst and 5 g of manganese chloride were added to 100 ml of eucalyptus oil. While, in this non-limiting example, the propionic acid has been used as organic acid component to prepare the composition, other organic acid (e.g. valeric, isovaleric, butyric, formic, benzoic, pelargonic, salicylic, malonic, citric, phthalic, tartaric, oxalic, malic, shikimic, fumaric, almond or cinnamic acid) may be used for preparation of the composition.

In this example, the catalyst used was a mixture of chromium (III) nitrate, nickel (II) sulfate and selenium sulfide mixed in a ratio of 0.1:100:0.1. The mixture was heated at boiling point, until the colour changed, under reflux for 60 minutes. The resulting mixture was allowed to cool at room temperature for 15 hours to obtain a clear solution, which was then filtered through filter paper.

### EXAMPLE 3

### A Combination of an Eucalyptus Oil (Eucalyptus globulus Labill.) with an Acid a Ratio of 80:1 and with Copper or Zinc

In this non-limiting example, the following two compositions were prepared:
a) composition IV—i.e., a composition of eucalyptus oil with a lactic acid and copper
b) composition V—i.e., a composition of eucalyptus oil with a lactic acid and zinc

Wherein, in order to prepare composition IV, 1 ml of lactic acid, 0.1 g of catalyst (i.e. a mixture of cobalt sulfate, ammonium molybdate and manganese sulfate mixed in ratio of 1:1:1) and 5 g of copper carbonate were added to 80 ml of eucalyptus oil. The mixture was heated at boiling point, until the colour changed, under reflux for 20 minutes. The mixture was then left to cool for 10 hours to obtain a clear solution (one, two or three phase solution). After this time, the reaction product was filtered through filter paper. Composition V was prepared analogously to composition IV, except that 1 g of zinc carbonate was used as the metal. Compositions IV and V were then analysed for their antiprotozoal properties analogously to Example 1. Results are presented in table 2.

TABLE 2
LD₅₀, LD₁₀₀ values for compositions IV and V, determined for selected protozoa.

| Sample | | | | Catalyst | Copper carbonate | Zinc carbonate | Eucalyptus oil | Composition | Composition |
|---|---|---|---|---|---|---|---|---|---|
| Protozoa | CH* | M** | Acid | solution | solution | solution | (Eucalyptus globulus Labill.) | IV | V |
| Euglena | LD₅₀: | LD₅₀: | LD₅₀: | LD₅₀: | LD₅₀: | LD₅₀: | LD₅₀: | LD₅₀: | LD₅₀: |
| gracilis | 0.05% | LD₁₀₀: | 0.5% | 0.05% | 0.15% | 0.1% | 0.1% | 0.04% | 0.035% |
| | LD₁₀₀: | | LD₁₀₀: | LD₁₀₀: | LD₁₀₀: | LD₁₀₀: | LD₁₀₀: | LD₁₀₀: | LD₁₀₀: |
| | 0.1% | | 1.1% | 0.1% | 0.25% | 0.3% | 0.2% | 0.07% | 0.08% |
| Gregarina | LD₅₀: | LD₅₀: | LD₅₀: | LD₅₀: | LD₅₀: | LD₅₀: | LD₅₀: | LD₅₀: | LD₅₀: |
| blattarum | LD₁₀₀: | 0.13% | 0.9% | 0.07% | 0.12% | 0.2% | 0.08% | 0.05% | 0.048% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.3% | 1.1% | 0.3% | 0.37% | 0.4% | 0.1% | 0.1% | 0.1% |
| Amoeba | LD₅₀: | LD₅₀: | LD₅₀: | LD₅₀: | LD₅₀: | LD₅₀: | LD₅₀: | LD₅₀: | LD₅₀: |
| proteus | 0.07% | 0.3% | 0.6% | 0.05% | 0.09% | 0.15% | 0.05% | 0.03% | 0.03% |
| | LD₁₀₀: | LD₁₀₀: | LD₁₀₀: | LD₁₀₀: | LD₁₀₀: | LD₁₀₀: | LD₁₀₀: | LD₁₀₀: | LD₁₀₀: |
| | 0.15% | 0.5% | 1% | 1% | 0.17% | 0.25% | 0.07% | 0.05% | 0.06% |
| Paramecium | LD₅₀: | LD₅₀: | LD₅₀: | LD₅₀: | LD₅₀: | LD₅₀: | LD₅₀: | LD₅₀: | LD₅₀: |
| caudatum | 0.001% | LD₁₀₀: | 0.8% | 0.8% | 0.35% | 0.3 | 0.1% | 0.07% | 0.08% |
| | LD₁₀₀: | | LD₁₀₀: | LD₁₀₀: | LD₁₀₀: | LD₁₀₀: | LD₁₀₀: | LD₁₀₀: | LD₁₀₀: |
| | 0.006% | | 1.25% | 1.25% | 0.5% | 0.5% | 0.3% | 0.1% | 0.12% |
| Trichomonas | LD₅₀: | LD₅₀: | LD₅₀: | LD₅₀: | LD₅₀: | LD₅₀: | LD₅₀: | LD₅₀: | LD₅₀: |
| hominis | LD₁₀₀: | 0.05% | 0.8% | 0.9% | 0.1% | 0.25% | 0.09% | 0.05% | 0.065% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.1% | 1% | 1.1% | 0.3% | 0.4% | 0.1% | 0.08% | 0.08% |

*CH—chloramphenicol;
**M—metronidazole

### EXAMPLE 4

### A Combination of a Cedar Oil (Cedrus atlantica (Endl.) Manetti ex Carrière, Cedrus deodara (Roxb. ex D. Don) G. Don, Cedrus libani A. Rich.) with Acid in a Ratio of 1:1 by Weight and with Copper or Zinc

In this non-limiting example, the following two compositions were prepared:
a) composition VI—i.e., a composition of cedar oil with a propionic acid and copper
b) composition VII—i.e., a composition of cedar oil with a propionic acid and zinc

In this non-limiting example, propionic acid is used, but other organic acids (e.g., acetic acid, lactic acid, etc.) may also be used. In order to prepare composition VI, 100 ml propionic acid, 0.6 g catalyst (which is cobalt sulfate, ammonium molybdate and manganese chloride mixed in a ratio of 1:10:20) and 5 g of copper carbonate were added to 100 ml of cedar oil. The mixture was heated at the boiling point, until the color changed, under reflux for 100 minutes. The mixture was then left to cool (for 20 hours) to obtain a clear solution (one, two or three phase solution). After this time, the reaction product was filtered through filter paper. Composition VII was prepared analogously to composition VI, except that zinc carbonate was used as the metal. Compositions VI and VII were then analysed for their antiprotozoal properties analogously to Example 1. Results were presented in Table 3. The results of the analysis showed that the killing and static activity in the compositions and after the reaction was higher than that of the individual substances in the reaction mixtures and complexes. Compositions VI and VII show many times stronger (potentiation) protozoal activity than each of these components separately. All the ingredients used in the compositions according to the invention are approved for both animal and human nutrition by the relevant directives and authorities, which, combined with their high efficacy, allows their use in the treatment and/or prevention of parasitoses in animals, caused by protozoa, in particular histomonadiasis (caused by Histomonas meleagridis), coccidiosis (caused by Eimeria), cryptosporidiosis (caused by Cryptosporidium), trichomonadiasis (caused by Trichomonas), babesiosis (caused by Babesia), or amoebiasis (caused by Amoeba).

TABLE 3
LD₅₀, LD₁₀₀ values for compositions VI and VII, determined for selected protozoa.

| Sample | | | | Catalyst | Copper carbonate | Zinc carbonate | | Composition | Composition |
|---|---|---|---|---|---|---|---|---|---|
| Protozoa | CH* | M** | Acid | solution | solution | solution | Cedar oil | VI | VII |
| Euglena | LD₅₀: | LD₅₀: | LD₅₀: | LD₅₀: | LD₅₀: | LD₅₀: | LD₅₀: | LD₅₀: | LD₅₀: |
| gracilis | 0.05% | LD₁₀₀: | 0.5% | 0.05% | 0.15% | 0.1% | 0.3% | 0.004% | 0.003% |
| | LD₁₀₀: | | LD₁₀₀: | LD₁₀₀: | LD₁₀₀: | LD₁₀₀: | LD₁₀₀: | LD₁₀₀: | LD₁₀₀: |
| | 0.1% | | 1.1% | 0.1% | 0.25% | 0.3% | 0.4% | 0.008% | 0.006% |
| Gregarina | LD₅₀: | LD₅₀: | LD₅₀: | LD₅₀: | LD₅₀: | LD₅₀: | LD₅₀: | LD₅₀: | LD₅₀: |
| blattarum | LD₁₀₀: | 0.13% | 0.9% | 0.07% | 0.12% | 0.2% | 0.1% | 0.003% | 0.004% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.3% | 1.1% | 0.3% | 0.37% | 0.4% | 0.2% | 0.006% | 0.005% |
| Amoeba | LD₅₀: | LD₅₀: | LD₅₀: | LD₅₀: | LD₅₀: | LD₅₀: | LD₅₀: | LD₅₀: | LD₅₀: |
| proteus | 0.07% | 0.3% | 0.6% | 0.05% | 0.09% | 0.15% | 0.08% | 0.004% | 0.002% |
| | LD₁₀₀: | LD₁₀₀: | LD₁₀₀: | LD₁₀₀: | LD₁₀₀: | LD₁₀₀: | LD₁₀₀: | LD₁₀₀: | LD₁₀₀: |
| | 0.15% | 0.5% | 1% | 1% | 0.17% | 0.25% | 0.1% | 0.005% | 0.005% |
| Paramecium | LD₅₀: | LD₅₀: | LD₅₀: | LD₅₀: | LD₅₀: | LD₅₀: | LD₅₀: | LD₅₀: | LD₅₀: |
| caudatum | 0.001% | LD₁₀₀: | 0.8% | 0.8% | 0.35% | 0.3 | 0.2% | 0.003% | 0.002% |
| | LD₁₀₀: | | LD₁₀₀: | LD₁₀₀: | LD₁₀₀: | LD₁₀₀: | LD₁₀₀: | LD₁₀₀: | LD₁₀₀: |
| | 0.006% | | 1.25% | 1.25% | 0.5% | 0.5% | 0.3% | 0.008% | 0.004% |
| Trichomonas | LD₅₀: | LD₅₀: | LD₅₀: | LD₅₀: | LD₅₀: | LD₅₀: | LD₅₀: | LD₅₀: | LD₅₀: |
| hominis | LD₁₀₀: | 0.08% | 0.8% | 0.9% | 0.9% | 0.25% | 0.25% | 0.09% | 0.025% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.15% | 1% | 1.1% | 1.1% | 0.4% | 0.4% | 0.15% | 0.07% |

*CH—chloramphenicol;
**M—metronidazole

### EXAMPLE 5

### A Combination of a Cedar Oil (Cedrus atlantica (Endl.) Manetti ex Carrière, Cedrus deodara (Roxb. ex D. Don) G. Don, Cedrus libani A. Rich.) with Acid in a Ratio of 1:80 by Weight and with Copper or Zinc

In this example, the following two compositions were prepared:
a) composition VIII—i.e., a composition of cedar oil with acetic acid and copper
b) composition IX—i.e., a composition of cedar oil with acetic acid and zinc

Although in this non-limiting example, copper or zinc were used in the composition, other metals, i.e. molybdenum, cobalt, nickel, chromium, zinc, bismuth, copper, manganese, selenium, iron, their salts, or oxides, can be used as the metal component for preparation the composition.

In order to prepare composition VIII, 80 ml of acetic acid, 1 g of catalyst (which is cobalt sulfate, ammonium molybdate and manganese sulfate mixed in ratio of 1:90:9) and 5 g of copper carbonate were added to 1 ml of cedar oil.

The mixture was heated at the boiling point, until the color changed, under reflux for 30 minutes. The mixture was then left to cool (for 10 hours) to obtain a clear solution (one, two or three phase solution). After this time, the reaction product was filtered through filter paper. Composition IX was prepared analogously to composition VIII, except that 1 g of zinc carbonate was used as the metal instead of copper carbonate. Compositions VIII and IX were then analysed for their antiprotozoal properties analogously to Example 1. Results were presented in Table 4.

TABLE 4
LD₅₀, LD₁₀₀ values for compositions VIII and IX, determined for selected protozoa.

| Sample | | | | Catalyst | Copper carbonate | Zinc carbonate | Cedar oil | Composition | Composition |
|---|---|---|---|---|---|---|---|---|---|
| Protozoa | CH* | M** | Acid | solution | solution | solution | (Cedrus sp.) | VIII | IX |
| Euglena | LD₅₀: | LD₅₀: | LD₅₀: | LD₅₀: | LD₅₀: | LD₅₀: | LD₅₀: | LD₅₀: | LD₅₀: |
| gracilis | 0.05% | LD₁₀₀: | 0.5% | 0.05% | 0.15% | 0.1% | 0.3% | 0.03% | 0.03% |
| | LD₁₀₀: | | LD₁₀₀: | LD₁₀₀: | LD₁₀₀: | LD₁₀₀: | LD₁₀₀: | LD₁₀₀: | LD₁₀₀: |
| | 0.1% | | 1.1% | 0.1% | 0.25% | 0.3% | 0.4% | 0.08% | 0.08% |
| Gregarina | LD₅₀: | LD₅₀: | LD₅₀: | LD₅₀: | LD₅₀: | LD₅₀: | LD₅₀: | LD₅₀: | LD₅₀: |
| blattarum | LD₁₀₀: | 0.13% | 0.9% | 0.07% | 0.12% | 0.2% | 0.1% | 0.05% | 0.055% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.3% | 1.1% | 0.3% | 0.37% | 0.4% | 0.2% | 0.08% | 0.08% |
| Amoeba | LD₅₀: | LD₅₀: | LD₅₀: | LD₅₀: | LD₅₀: | LD₅₀: | LD₅₀: | LD₅₀: | LD₅₀: |
| proteus | 0.07% | 0.3% | 0.6% | 0.05% | 0.09% | 0.15% | 0.08% | 0.03% | 0.02% |
| | LD₁₀₀: | LD₁₀₀: | LD₁₀₀: | LD₁₀₀: | LD₁₀₀: | LD₁₀₀: | LD₁₀₀: | LD₁₀₀: | LD₁₀₀: |
| | 0.15% | 0.5% | 1% | 1% | 0.17% | 0.25% | 0.1% | 0.05% | 0.05% |
| Paramecium | LD₅₀: | LD₅₀: | LD₅₀: | LD₅₀: | LD₅₀: | LD₅₀: | LD₅₀: | LD₅₀: | LD₅₀: |
| caudatum | 0.001% | LD₁₀₀: | 0.8% | 0.8% | 0.35% | 0.3 | 0.2% | 0.07% | 0.05% |
| | LD₁₀₀: | | LD₁₀₀: | LD₁₀₀: | LD₁₀₀: | LD₁₀₀: | LD₁₀₀: | LD₁₀₀: | LD₁₀₀: |
| | 0.006% | | 1.25% | 1.25% | 0.5% | 0.5% | 0.3% | 0.15% | 0.17% |
| Trichomanas | LD₅₀: | LD₅₀: | LD₅₀: | LD₅₀: | LD₅₀: | LD₅₀: | LD₅₀: | LD₅₀: | LD₅₀: |
| hominis | LD₁₀₀: | 0.08% | 0.8% | 0.9% | 0.9% | 0.25% | 0.25% | 0.06% | 0.07% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.15% | 1% | 1.1% | 1.1% | 0.4% | 0.4% | 0.08% | 0.08% |

*CH—chloramphenicol;
**M—metronidazole

### EXAMPLE 6

### A Combination of Yarrow Oil (Achillea millefolium Linne) with a Mixture of Acids in a Ratio of 1:1 by Weight and with Copper or Zinc

In this non-limiting example, the following two compositions were prepared:
a) composition X—i.e., a composition of yarrow oil with a mixture of acids and copper
b) composition XI—i.e., a composition of yarrow oil with a mixture of acids and zinc carbonate

Although, in this non-limiting example, copper or zinc were used as a metal component, other metals such as molybdenum, cobalt, nickel, chromium, zinc, bismuth, copper, manganese, selenium, iron, their salts, or oxides can be used in the preparation of composition. In order to prepare composition X, 100 ml of an acid mixture (containing acetic, propionic, lactic and formic acids mixed in a ratio of 1:1:1:1), 0.6 g of catalyst (which is cobalt sulfate, ammonium molybdate and manganese chloride mixed in a ratio of 1:1:1) and 5 g of copper carbonate were added to 100 ml of yarrow oil. The mixture was heated at boiling point, until the colour changed, under reflux for 120 minutes. The mixture was then left to cool (for 24 hours) to obtain a clear solution (one, two or three phase solution). After this time, the reaction product was filtered through filter paper. Composition XI was prepared analogously to composition X, except that 5 g of zinc carbonate was used as the metal instead of copper carbonate. Compositions X and XI were then analysed for their antiprotozoal properties. Results were presented in Table 5. The results of the analysis showed that the killing and static activity in the compositions and after the reaction was higher than that of the individual substances in the reaction mixtures and complexes. All the ingredients used in the compositions are approved for both animal and human nutrition by the relevant directives and authorities, which, combined with their high efficacy, allows their use in the treatment and/or prevention of parasitoses in animals, caused by protozoa, in particular histomonadiasis (caused by Histomonas meleagridis), coccidiosis (caused by Eimeria), cryptosporidiosis (caused by Cryptosporidium), trichomonadiasis (caused by Trichomonas), babesiosis (caused by Babesia), or amoebiasis (caused by Amoeba).

TABLE 5
LD₅₀, LD₁₀₀ values for compositions X and XI, determined for selected protozoa.

| Sample | | | Mixture of | Catalyst | Copper carbonate | Zinc carbonate | Yarrow oil | Composition | Composition |
|---|---|---|---|---|---|---|---|---|---|
| Protozoa | CH* | M** | acids | solution | solution | solution | (Achillea millefolium L.) | X | XI |
| Euglena | LD₅₀: | LD₅₀: | LD₅₀: | LD₅₀: | LD₅₀: | LD₅₀: | LD₅₀: | LD₅₀: | LD₅₀: |
| gracilis | 0.05% | LD₁₀₀: | 0.5% | 0.05% | 0.15% | 0.1% | 0.08% | 0.035% | 0.025% |
| | LD₁₀₀: | | LD₁₀₀: | LD₁₀₀: | LD₁₀₀: | LD₁₀₀: | LD₁₀₀: | LD₁₀₀: | LD₁₀₀: |
| | 0.1% | | 1.1% | 0.1% | 0.25% | 0.3% | 0.1% | 0.055% | 0.035% |
| Gregarina | LD₅₀: | LD₅₀: | LD₅₀: | LD₅₀: | LD₅₀: | LD₅₀: | LD₅₀: | LD₅₀: | LD₅₀: |
| blattarum | LD₁₀₀: | 0.13% | 0.9% | 0.07% | 0.12% | 0.2% | 0.1% | 0.025% | 0.065% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.3% | 1.1% | 0.3% | 0.37% | 0.4% | 0.2% | 0.035% | 0.075% |
| Amoeba | LD₅₀: | LD₅₀: | LD₅₀: | LD₅₀: | LD₅₀: | LD₅₀: | LD₅₀: | LD₅₀: | LD₅₀: |
| proteus | 0.07% | 0.3% | 0.6% | 0.05% | 0.09% | 0.15% | 0.09% | 0.065% | 0.045% |
| | LD₁₀₀: | LD₁₀₀: | LD₁₀₀: | LD₁₀₀: | LD₁₀₀: | LD₁₀₀: | LD₁₀₀: | LD₁₀₀: | LD₁₀₀: |
| | 0.15% | 0.5% | 1% | 1% | 0.17% | 0.25% | 0.1% | 0.075% | 0.075% |
| Paramecium | LD₅₀: | LD₅₀: | LD₅₀: | LD₅₀: | LD₅₀: | LD₅₀: | LD₅₀: | LD₅₀: | LD₅₀: |
| caudatum | 0.001% | LD₁₀₀: | 0.8% | 0.8% | 0.35% | 0.3 | 0.1% | 0.055% | 0.075% |
| | LD₁₀₀: | | LD₁₀₀: | LD₁₀₀: | LD₁₀₀: | LD₁₀₀: | LD₁₀₀: | LD₁₀₀: | LD₁₀₀: |
| | 0.006% | | 1.25% | 1.25% | 0.5% | 0.5% | 0.3% | 0.065% | 0.095% |
| Trichomonas | LD₅₀: | LD₅₀: | LD₅₀: | LD₅₀: | LD₅₀: | LD₅₀: | LD₅₀: | LD₅₀: | LD₅₀: |
| hominis | LD₁₀₀: | 0.08% | 0.8% | 0.9% | 0.9% | 0.25% | 0.09% | 0.025% | 0.045% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.15% | 1% | 1.1% | 1.1% | 0.4% | 0.1% | 0.035% | 0.075% |

*CH—chloramphenicol;
**M—metronidazole

### EXAMPLE 7

### A Combination of a Wormwood Oil (Artemisia absinthium Linne) with a Mixture of Acids in a Ratio of 1:1 by Weight and with Copper or Zinc

In this example following two compositions were prepared:
a) composition XII—i.e., a composition of wormwood oil with a mixture of acids and copper
b) composition XIII—i.e., a composition of wormwood oil with a mixture of acids and zinc

In order to prepare composition XII, 100 ml mixture of acids (containing acetic acid, propionic acid, lactic acid and formic acid mixed in a ratio of 1:1:1:1), 0.6 g of catalyst (which is cobalt sulfate, ammonium molybdate and manganese sulfate mixed in a ratio of 1:1:1) and 5 g copper carbonate were added to 100 ml of wormwood oil. The mixture was heated at boiling point, until the colour changed, under reflux for 90 minutes. The mixture was then left to cool (for 12 hours) to obtain a clear solution (one, two or three phase solution). After this time, the reaction product was filtered through filter paper.

Composition XIII was prepared analogously to composition XII, except that 5 g of zinc carbonate was added instead of copper carbonate. Compositions XII and XIII were then analysed for their antiprotozoal properties analogously to Example 1. Results are presented in Table 6.

The results obtained from the protozoal activity test are presented in Table 6. The results of the analysis showed that the killing and static activity in the compositions and after the reaction was higher than that of the individual substances in the reaction mixtures and complexes. All the ingredients used in the compositions are approved for both animal and human nutrition by the relevant directives and authorities, which, combined with their high efficacy, allows their use in the treatment and/or prevention of parasitoses in animals, caused by protozoa, in particular histomonadiasis (caused by Histomonas meleagridis), coccidiosis (caused by Eimeria), cryptosporidiosis (caused by Cryptosporidium), trichomonadiasis (caused by Trichomonas), babesiosis (caused by Babesia), or amoebiasis (caused by Amoeba).

TABLE 6
LD₅₀, LD₁₀₀ values for compositions XII and XIII, determined for selected protozoa.

| Sample | | | Mixture of | Catalyst | Copper carbonate | Zinc carbonate | Wormwood oil (Artemisia | Composition | Composition |
|---|---|---|---|---|---|---|---|---|---|
| Protozoa | CH* | M** | acids | solution | solution | solution | absinthium Linne) | XII | XIII |
| Euglena | LD₅₀: | LD₅₀: | LD₅₀: | LD₅₀: | LD₅₀: | LD₅₀: | LD₅₀: | LD₅₀: | LD₅₀: |
| gracilis | 0.05% | LD₁₀₀: | 0.5% | 0.05% | 0.15% | 0.1% | 0.1% | 0.004% | 0.002% |
| | LD₁₀₀: | | LD₁₀₀: | LD₁₀₀: | LD₁₀₀: | LD₁₀₀: | LD₁₀₀: | LD₁₀₀: | LD₁₀₀: |
| | 0.1% | | 1.1% | 0.1% | 0.25% | 0.3% | 0.3% | 0.005% | 0.004% |
| Gregarina | LD₅₀: | LD₅₀: | LD₅₀: | LD₅₀: | LD₅₀: | LD₅₀: | LD₅₀: | LD₅₀: | LD₅₀: |
| blattarum | LD₁₀₀: | 0.13% | 0.9% | 0.07% | 0.12% | 0.2% | 0.1% | 0.002% | 0.004% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.3% | 1.1% | 0.3% | 0.37% | 0.4% | 0.2% | 0.005% | 0.006% |
| Amoeba | LD₅₀: | LD₅₀: | LD₅₀: | LD₅₀: | LD₅₀: | LD₅₀: | LD₅₀: | LD₅₀: | LD₅₀: |
| proteus | 0.07% | 0.3% | 0.6% | 0.05% | 0.09% | 0.15% | 0.1% | 0.002% | 0.005% |
| | LD₁₀₀: | LD₁₀₀: | LD₁₀₀: | LD₁₀₀: | LD₁₀₀: | LD₁₀₀: | LD₁₀₀: | LD₁₀₀: | LD₁₀₀: |
| | 0.15% | 0.5% | 1% | 1% | 0.17% | 0.25% | 0.2% | 0.004% | 0.007% |
| Paramecium | LD₅₀: | LD₅₀: | LD₅₀: | LD₅₀: | LD₅₀: | LD₅₀: | LD₅₀: | LD₅₀: | LD₅₀: |
| caudatum | 0.001% | LD₁₀₀: | 0.8% | 0.8% | 0.35% | 0.3% | 0.4% | 0.006% | 0.003% |
| | LD_{100:} | | LD₁₀₀: | LD₁₀₀: | LD₁₀₀: | LD₁₀₀: | LD₁₀₀: | LD₁₀₀: | LD₁₀₀: |
| | 0.006% | | 1.25% | 1.25% | 0.5% | 0.5% | 0.5% | 0.007% | 0.005% |
| Trichomonas | LD₅₀: | LD₅₀: | LD₅₀: | LD₅₀: | LD₅₀: | LD₅₀: | LD₅₀: | LD₅₀: | LD₅₀: |
| hominis | LD₁₀₀: | 0.08% | 0.8% | 0.9% | 0.9% | 0.25% | 0.09% | 0.001% | 0.004% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.15% | 1% | 1.1% | 1.1% | 0.4% | 0.1% | 0.002% | 0.007% |

*CH—chloramphenicol;
**M—metronidazole

### EXAMPLE 8

### A Combination of Origanum Oil (Origanum vulgare L.) with a Mixture of Acids in a Ratio of 1:1 by Weight and with Copper or Zinc

In this example the following two compositions were prepared:
a) composition XIV—i.e., a composition of Origanum oil with a mixture of acids and copper
b) composition XV—i.e., a composition of Origanum oil with a mixture of acids and zinc

In order to prepare composition XIV, 100 ml mixture of acids (containing acetic acid, propionic acid, lactic acid and formic acid mixed in a ratio of 1:1:1:1), 0.6 g of catalyst (which is cobalt sulfate, ammonium molybdate and manganese sulfate mixed in a ratio of 1:1:1) and 5 g copper carbonate were added to 100 ml of Origanum oil. The mixture was heated at the boiling point, until the color changed, under reflux for 20 minutes. The mixture was then left to cool (for 10 hours) to obtain a clear solution (one, two or three phase solution). After this time, the reaction product was filtered through filter paper.

Composition XV was prepared analogously to composition XIV, except that 5 g of zinc carbonate was added instead of copper carbonate. Compositions XIV and XV were then analysed for their antiprotozoal properties analogously to Example 1. Results are presented in Table 7.

TABLE 7
LD₅₀, LD₁₀₀ values for compositions XIV and XV, determined for selected protozoa.

| Sample | | | Mixture of | Catalyst | Copper carbonate | Zinc carbonate | Origanum | Composition | Composition |
|---|---|---|---|---|---|---|---|---|---|
| Protozoa | CH* | M** | acids | solution | solution | solution | oil | XIV | XV |
| Euglena | LD₅₀: | LD₅₀: | LD₅₀: | LD₅₀: | LD₅₀: | LD₅₀: | LD₅₀: | LD₅₀: | LD₅₀: |
| gracilis | 0.05% | LD₁₀₀: | 0.5% | 0.05% | 0.15% | 0.1% | 0.5% | 0.035% | 0.03% |
| | LD₁₀₀: | | LD₁₀₀: | LD₁₀₀: | LD₁₀₀: | LD₁₀₀: | LD₁₀₀: | LD₁₀₀: | LD₁₀₀: |
| | 0.1% | | 1.1% | 0.1% | 0.25% | 0.3% | 0.6% | 0.07% | 0.06% |
| Gregarina | LD₅₀: | LD₅₀: | LD₅₀: | LD₅₀: | LD₅₀: | LD₅₀: | LD₅₀: | LD₅₀: | LD₅₀: |
| blattarum | LD₁₀₀: | 0.3% | 0.9% | 0.07% | 0.12% | 0.2% | 0.4% | 0.05% | 0.03% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.3% | 1.1% | 0.3% | 0.37% | 0.4% | 0.5% | 0.08% | 0.04% |
| Amoeba | LD₅₀: | LD₅₀: | LD₅₀: | LD₅₀: | LD₅₀: | LD₅₀: | LD₅₀: | LD₅₀: | LD₅₀: |
| proteus | 0.07% | 0.3% | 0.6% | 0.05% | 0.09% | 0.15% | 0.5% | 0.04% | 0.03% |
| | LD₁₀₀: | LD₁₀₀: | LD₁₀₀: | LD₁₀₀: | LD₁₀₀: | LD₁₀₀: | LD₁₀₀: | LD₁₀₀: | LD₁₀₀: |
| | 0.15% | 0.5% | 1% | 1% | 0.17% | 0.25% | 0.7% | 0.09% | 0.08% |
| Paramecium | LD₅₀: | LD₅₀: | LD₅₀: | LD₅₀: | LD₅₀: | LD₅₀: | LD₅₀: | LD₅₀: | LD₅₀: |
| caudatum | 0.001% | LD₁₀₀: | 0.8% | 0.8% | 0.35% | 0.3% | 0.4% | 0.1% | 0.09% |
| | LD₁₀₀: | | LD₁₀₀: | LD₁₀₀: | LD₁₀₀: | LD₁₀₀: | LD₁₀₀: | LD₁₀₀: | LD₁₀₀: |
| | 0.006% | | 1.25% | 1.25% | 0.5% | 0.5% | 0.5% | 0.3% | 0.25% |
| Trichomonas | LD₅₀: | LD₅₀: | LD₅₀: | LD₅₀: | LD₅₀: | LD₅₀: | LD₅₀: | LD₅₀: | LD₅₀: |
| hominis | LD₁₀₀: | 0.08% | 0.8% | 0.9% | 0.9% | 0.25% | 0.2% | 0.01% | 0.05% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.15% | 1% | 1.1% | 1.1% | 0.4% | 0.3% | 0.06% | 0.07% |

*CH—chloramphenicol;
**M—metronidazole

### EXAMPLE 9

### A Combination of a Lavender Oil (Lavandula angustifolia Miller) with a Mixture of Acids in a Ratio of 1:1 by Weight and with Copper or Zinc

In this non-limiting example, lavender oil was used to prepare the compositions. However, another essential oil selected from the group consisting of: cedar oil, tea oil, cardamom oil, wormwood oil, cubeb oil, eucalyptus oil, mint oil, juniper oil, cypress oil, lemongrass oil, spruce oil, pine oil, fir oil, Douglas fir oil, caraway oil, cumin oil, patchouli oil, sage oil, Inula oil, tansy oil, angelica oil calamus oil, ginger oil, thyme oil, Origanum oil, rosemary oil, nutmeg oil, coriander oil, geranium oil, carrot seed oil, yarrow herb or flower oil can be used in composition according to invention as an essential oil component

In this example the following two compositions were prepared:
a) composition XVI—i.e., a composition of lavender oil with a mixture of acids and copper
b) composition XVII—i.e., a composition of lavender oil with a mixture of acids and zinc

Wherein, in order to prepare composition XVI, 100 ml mixture of acids (containing acetic acid, propionic acid, lactic acid and formic acid mixed in a ratio of 1:1:1:1), 0.3 g of catalyst (which is cobalt sulfate, ammonium molybdate and manganese sulfate mixed in a ratio of 1:1:1) and 5 g copper carbonate were added to 100 ml of lavender oil. The mixture was heated at boiling point, until the colour changed, under reflux for 120 minutes. The mixture was then left to cool (for 24 hours) to obtain a clear solution (one, two or three phase solution). After this time, the reaction product was filtered through filter paper.

Composition XVII was prepared analogously to composition XVI, except that 5 g of zinc carbonate was added instead of copper carbonate. Compositions XVI and XVII were then analysed for their antiprotozoal properties analogously to Example 1. Results are presented in Table 8. The results of the analysis showed that the killing and static activity in complex systems and after the reaction was higher than that of the substances separately, included in the reaction mixtures and complexes.

TABLE 8
LD₅₀, LD₁₀₀ values for compositions XVI and XVII, determined for selected protozoa.

| Sample | | | Mixture of | Catalyst | Copper carbonate | Zinc carbonate | Lavender oil (Lavandula | Composition | Composition |
|---|---|---|---|---|---|---|---|---|---|
| Protozoa | CH* | M** | acids | solution | solution | solution | angustifolia Miller) | XVI | XVII |
| Euglena | LD₅₀: | LD₅₀: | LD₅₀: | LD₅₀: | LD₅₀: | LD₅₀: | LD₅₀: | LD₅₀: | LD₅₀: |
| gracilis | 0.05% | LD₁₀₀: | 0.5% | 0.05% | 0.15% | 0.1% | 0.1% | 0.025% | 0.025% |
| | LD₁₀₀: | | LD₁₀₀: | LD₁₀₀: | LD₁₀₀: | LD₁₀₀: | LD₁₀₀: | LD₁₀₀: | LD₁₀₀: |
| | 0.1% | | 1.1% | 0.1% | 0.25% | 0.3% | 0.2% | 0.045% | 0.055% |
| Gregarina | LD₅₀: | LD₅₀: | LD₅₀: | LD₅₀: | LD₅₀: | LD₅₀: | LD₅₀: | LD₅₀: | LD₅₀: |
| blattarum | LD₁₀₀: | 0.13% | 0.9% | 0.07% | 0.12% | 0.2% | 0.1% | 0.055% | 0.055% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.3% | 1.1% | 0.3% | 0.37% | 0.4% | 0.2% | 0.075% | 0.085% |
| Amoeba | LD₅₀: | LD₅₀: | LD₅₀: | LD₅₀: | LD₅₀: | LD₅₀: | LD₅₀: | LD₅₀: | LD₅₀: |
| proteus | 0.07% | 0.3% | 0.6% | 0.05% | 0.09% | 0.15% | 0.1% | 0.025% | 0.025% |
| | LD₁₀₀: | LD₁₀₀: | LD₁₀₀: | LD₁₀₀: | LD₁₀₀: | LD₁₀₀: | LD₁₀₀: | LD₁₀₀: | LD₁₀₀: |
| | 0.15% | 0.5% | 1% | 1% | 0.17% | 0.25% | 0.2% | 0.055% | 0.065% |
| Paramecium | LD₅₀: | LD₅₀: | LD₅₀: | LD₅₀: | LD₅₀: | LD₅₀: | LD₅₀: | LD₅₀: | LD₅₀: |
| caudatum | 0.001% | LD₁₀₀: | 0.8% | 0.8% | 0.35% | 0.3% | 0.1% | 0.025% | 0.045% |
| | LD₁₀₀: | | LD₁₀₀: | LD₁₀₀: | LD₁₀₀: | LD₁₀₀: | LD₁₀₀: | LD₁₀₀: | LD₁₀₀: |
| | 0.006% | | 1.25% | 1.25% | 0.5% | 0.5% | 0.2% | 0.045% | 0.065% |
| Trichomonas | LD₅₀: | LD₅₀: | LD₅₀: | LD₅₀: | LD₅₀: | LD₅₀: | LD₅₀: | LD₅₀: | LD₅₀: |
| hominis | LD₁₀₀: | 0.08% | 0.8% | 0.9% | 0.9% | 0.25% | 0.1% | 0.055% | 0.085% |
| | | LD₁₀₀: | LD₁₀₀: | LD₁₀₀: | LD₁₀₀: | LD₁₀₀: | LD₁₀₀: | LD₁₀₀: | LD₁₀₀: |
| | | 0.15% | 1% | 1.1% | 1.1% | 0.4% | 0.2% | 0.075% | 0.15% |

*CH—chloramphenicol;
**M—metronidazole

1.-8. (canceled)

9. A veterinary composition to treat and/or prevent protozoan diseases in animals, the composition comprising:
an essential oil including cedar oil, tea oil, cardamom oil, lavender oil, cubeb oil, eucalyptus oil, mint oil, juniper oil, cypress oil, lemongrass oil, spruce oil, pine oil, fir oil, Douglas fir oil, wormwood oil, caraway oil, cumin oil, patchouli oil, sage oil, Inula oil, tansy oil, angelica oil calamus oil, ginger oil, thyme oil, Origanum oil, rosemary oil, nutmeg oil, coriander oil, geranium oil, carrot seed oil, yarrow herb, or flower oil, the essential oil present in a form of a complex with an organic acid or a mixture of organic acids, and a metal, the organic acid or mixture of organic acids including valerian, isovalerian, lactic, butyric, acetic, propionic, formic, benzoic, pelargonic, salicylic, malonic, citric, phthalic, tartaric, oxalic, malic, shikimic, fumaric, almond, cinnamic, or derivatives thereof, and a metal including molybdenum, cobalt, nickel, chromium, zinc, bismuth, copper, manganese, selenium, iron, or salts or oxides thereof.

10. The composition of claim 9, wherein the complex is with the mixture of organic acids and the mixture includes acetic acid, propionic acid, lactic acid and formic acid.

11. The composition of claim 10, wherein acids in the mixture of organic acids are mixed at a ratio of 1:1:1:1:1.

12. The composition of claim 10, wherein the essential oil includes eucalyptus oil, cedar oil, yarrow oil, wormwood oil, Origanum oil, or lavender oil.

13. The composition of claim 9, wherein the essential oil includes eucalyptus oil.

14. The composition of claim 9, wherein the essential oil includes cedar oil.

15. The composition of claim 9, wherein the essential oil includes yarrow oil.

16. The composition of claim 9, wherein the essential oil includes wormwood oil.

17. The composition of claim 9, wherein the essential oil includes Origanum oil.

18. The composition of claim 9, wherein the essential oil includes lavender oil.

19. A method of manufacturing a veterinary composition to treat and/or prevent protozoan diseases in animals, the method comprising:
a) mixing at least one essential oil, and an organic acid or a mixture of organic acids at a ratio of 80:1 to 1:80 by weight, the essential oil including cedar oil, tea oil, cardamom oil, lavender oil, cubeb oil, eucalyptus oil, mint oil, juniper oil, cypress oil, lemongrass oil, spruce oil, pine oil, fir oil, Douglas fir oil, woanwood oil, caraway oil, cumin oil, patchouli oil, sage oil, Inula oil, tansy oil, angelica oil calamus oil, ginger oil, thyme oil, Origanum oil, rosemary oil, nutmeg oil, coriander oil, geranium oil, carrot seed oil, yarrow herb, or flower oil and the organic acid or the mixture of organic acids including valerian, isovalerian, lactic, butyric, acetic, propionic, formic, benzoic, pelargonic, salicylic, malonic, citric, phthalic, tartaric, oxalic, malic, shilcimic, fumaric, almond, cinnamic, or derivatives thereof;
b) adding a catalyst and a metal including molybdenum, cobalt, nickel, chromium, zinc, bismuth, copper, manganese, selenium, iron, or salts or oxides thereof to form a mixture;
c) heating the mixture to form a reaction product;
d) allowing the reaction product to cool; and
e) filtering the reaction product.

20. The method of claim 19, wherein the at least one essential oil is mixed with the organic acid or the mixture of organic acids at a ratio of 1:1 by weight.

21. The method of claim 19, wherein the at least one essential oil is mixed with the mixture of organic acids and the mixture of organic acids includes acetic acid, propionic acid, lactic acid and formic acid.

22. The method of claim 21, wherein acids in the mixture of organic acids are mixed at a ratio of 1:1:1:1.

23. The method of claim 19, wherein the catalyst includes cobalt sulphate, ammonium molybdate and manganese chloride or sulphate.

24. The method of claim 19, wherein the mixture is heated under reflux.

25. The method of claim 19, wherein the mixture is heated for 20 to 120 minutes.

26. The method of claim 19, wherein the reaction product is cooled for 10 to 24 hours.

27. The method of claim 19, wherein the mixture is heated to a boiling point of the mixture.

28. The method of claim 19, wherein the essential oil includes eucalyptus oil, cedar oil, yarrow oil, wormwood oil, Origanum oil, or lavender oil.
